(19) United States
(12) Patent Application Publication
(10) Pub. No.: US 2005/0070955 A1
(43) Pub. Date: Mar. 31, 2005

(54) Articulable forceps

(75) Inventor: Terrence R. Young, East Taunton, MA (US)

Correspondence Address: DARBY & DARBY P.C., P. O. BOX 5257, NEW YORK, NY, 10150-5257, US

(73) Assignee: Codman & Shurtleff, Inc., Raynham, MA (US)

(21) Appl. No.: 10/676,337

(22) Filed: Sep. 30, 2003

(51) Int. Cl. A61B 17/50

(52) U.S. Cl. 606/210

(57) ABSTRACT

Forceps include a first and a second blade, both of which have an inner surface, an outer surface, a tip end, and a proximal end. The forceps also include a blade adjustment joint disposed adjacent to the proximal ends of the first and the second blades and both blades are rotateably connected to the blade adjustment joint. The blade adjustment joint selectively allows the first and second blades to rotate independently of each other or prevents both blades from rotating independently of each other. The blade adjustment joint permits the first blade to rotate or pivot in relation to the second blade and visa versa, so the tips of the forceps can be realigned simply and easily. Additionally, the blade adjustment joint in one condition prevents the first blade and second blade from rotating, locking the blades in position for use.

### BACKGROUND OF THE INVENTION

1. Field of the Invention

This invention relates to forceps in which each blade or a portion of each blade can be individually articulated through a range of angles, regardless of whether the tips of the blades are in contact or not. Additionally, the blades can be articulated with no or minimal pressure exerted on the articulating portion of the blade while the forceps are in use.

2. Discussion of the Related Art

Angled forceps and surgical instruments are well known in the art. FIGS. 1A and 1B illustrate prior art forceps 10 where blades 12, having a tip sections 14 and a handle section 16, can be manufactured straight or with a preset angle X between a horizontal axis 18 and a center line 20 of tip section 14. Since the angle X is preset when the forceps are manufactured, it cannot be altered by a user. Multiple forceps are required to allow the user a full range of angles while performing medical procedures. Additionally, as illustrated in FIG. 2 in which the forecep tips have been brought together, all prior art forceps become misaligned after repeated use, especially fine tip forceps. Misalignment of the tips of the blades reduces the users ability to secure a firm grip on an object and increases the user's hand fatigue.

Numerous other prior art references disclose surgical instruments, including dissectors and clamps, wherein the operating end can be angled with respect to the shaft of the instrument. However, some prior art devices permit the articulation of the tip at a fixed angle to the elongate shaft from which the tip extends. For example, U.S. Pat. No. 5,411,519 to Tovey et al. discloses a surgical apparatus with hinged jaws where the jaws are either designed with a preset angle or once the jaws are fully deployed, they are biased to a preset angle. Additionally, Tovey et al. does not lock the jaws in position, the jaws are only biased into position using a spring and the angle can be changed if the user applies pressure on the jaws.

Other prior art devices allow for a variable articulated tip. However, in those devices both blades must be articulated together and set to the same angle. For example, U.S. Pat. No. 5,374,277 to Hassler, allows the tip of the surgical instrument to articulated through multiple angles, but both end effectors 60, 70 move through the same angle. Thus, while allowing the user to alter the angle of the tip, the tip will still suffer the same misalignment problem described above.

Further, U.S. Pat. No. 5,649,957 to Levin discloses an articulated dissector, wherein a tip 20 has jaws 22, 24 which can be angled about pins 28. However, to change the angle of the jaws while the dissector is in use, the surgeon must press the distal sections of the jaws against a body wall or structure. The difficulty in articulating jaws 22, 24 in such a fashion is it may not be possible to press the tool against a body section or the pressure required to change the angle of the tip may damage the body section. Additionally, if Levin's tip is designed to require minimum pressure to change the angle of the tip, the tip will not be secured in the angle set. Since only minimal pressure will be required to move the tip, the tip will move during use.

Thus, there is still a need in the art for a surgical instrument wherein the tip of each blade is separately articulate, it requires no or minimal pressure to articulate the tip and the tip is secured at the set angle.

### SUMMARY OF THE INVENTION

The invention provides forceps that include a first blade and a second blade, both of which have an inner surface, an outer surface, a tip end, and a proximal end. The forceps also include a blade adjustment joint disposed proximate to the proximal ends of the first and the second blades and to which the first and second blades are rotateably connected. The blade adjustment joint selectively allows the first and the second blades to rotate independently of each other or prevents the first and second blades from rotating independently of each other so the tips of the forceps can be realigned simply and easily. Additionally, the blade adjustment joint can be set to prevent the first blade and second blade from rotating. Thus locking the blades in position for use. The blade adjustment joint can be manufactured to permit unrestricted rotation and completely restrict rotation of the first and second blades.

The blade adjustment joint includes a bolt having a head end and a threaded end, and a nut having a contact surface threadably engagable with the threaded end of the bolt. Additionally, the first and second blades include openings proximate to both of their proximal ends. The threaded end of the bolt passes through the first and second blade openings and the head end engages the outer surface of the first blade. Thus, when the nut is screwed down, the threaded end of the bolt selectively prevents both the first and second blades from rotating. Once the nut is loosened, both the first and second blades can freely rotate.

In one embodiment, the contact surface frictionally engages the outer surface of the second blade, and the head end can frictionally engage the outer surface of the first blade. The frictional engagement prevents the blades from rotating.

The forceps can also be electrically charged as electro-surgical forceps. In this configuration, either one or both blades can be electrically charged. The electro-surgical forceps include at least one electrical terminal electrically connected to at least one of the first and/or second blades. The electro-surgical forceps require an insulator disposed between the first blade and the second blade to electrically isolate the first blade from second blade. Additionally, the bolt and nut can be manufactured from a non-electrically conductive material, e.g. plastic.

### BRIEF DESCRIPTION OF THE DRAWING FIGURES

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIGS. 1A and 1B are perspective views of prior art forceps;

FIG. 2 is a magnified view of misaligned tips of the prior art forceps;

FIG. 3 is a perspective view of the forceps of the present invention;

FIG. 4A is a cross sectional view of FIG. 3 along lines 4-4 illustrating an embodiment of the blade adjustment joint;

FIG. 4B is an exploded cross sectional of the blade adjustment joint of FIG. 4A along line 4-4;

FIGS. 4C and 4D are cross sectional views of the blade adjustment joint of FIG. 4A along line 4-4 illustrating another embodiment of the blade adjustment joint;

FIGS. 4E and 4F are cross sectional views of the blade adjustment joint of FIG. 4A along line 4-4 illustrating another embodiment of the blade adjustment joint;

FIG. 4G is a cross sectional view of the blade adjustment joint of FIG. 3 along line 4-4 illustrating another embodiment thereof;

FIG. 5 is a perspective view of electro-surgical forceps of the present invention:

FIG. 6 is a cross sectional view of the blade adjustment joint of FIG. 5 along line 6-6 illustrating an embodiment of the blade adjustment joint;

FIG. 7A is a perspective view of a forceps with a tip adjustment joint;

FIG. 7B is a cross sectional view of the tip adjustment joint of FIG. 7A;

FIG. 7C is a cross sectional view of another embodiment of the tip adjustment joint of FIG. 7A;

FIG. 8 top view of an embodiment of a blade angle adjustment joint;

FIG. 9 is a cross sectional view of FIG. 8 along line 8-8;

FIG. 10 is a top view of another embodiment of a blade adjustment joint;

FIG. 11 is a top view of another embodiment of a blade adjustment joint; and

FIG. 12 is a cross sectional view of the blade adjustment joint of FIG. 11 along line 11-11.

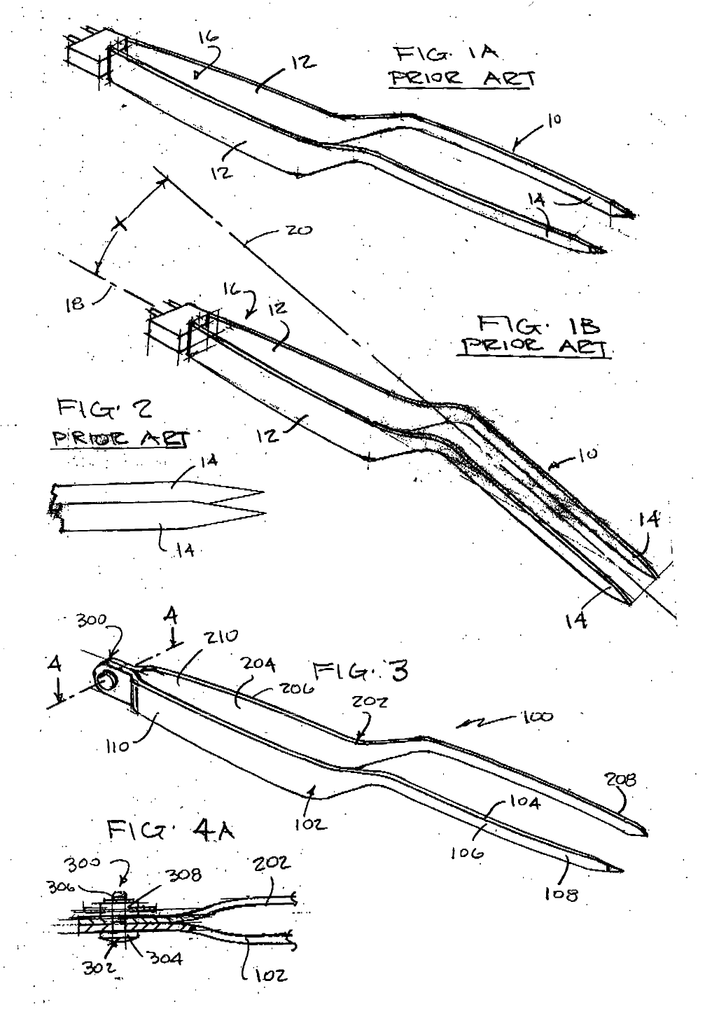

### DETAILED DESCRIPTION OF THE PREFERRED EMBODIMENT

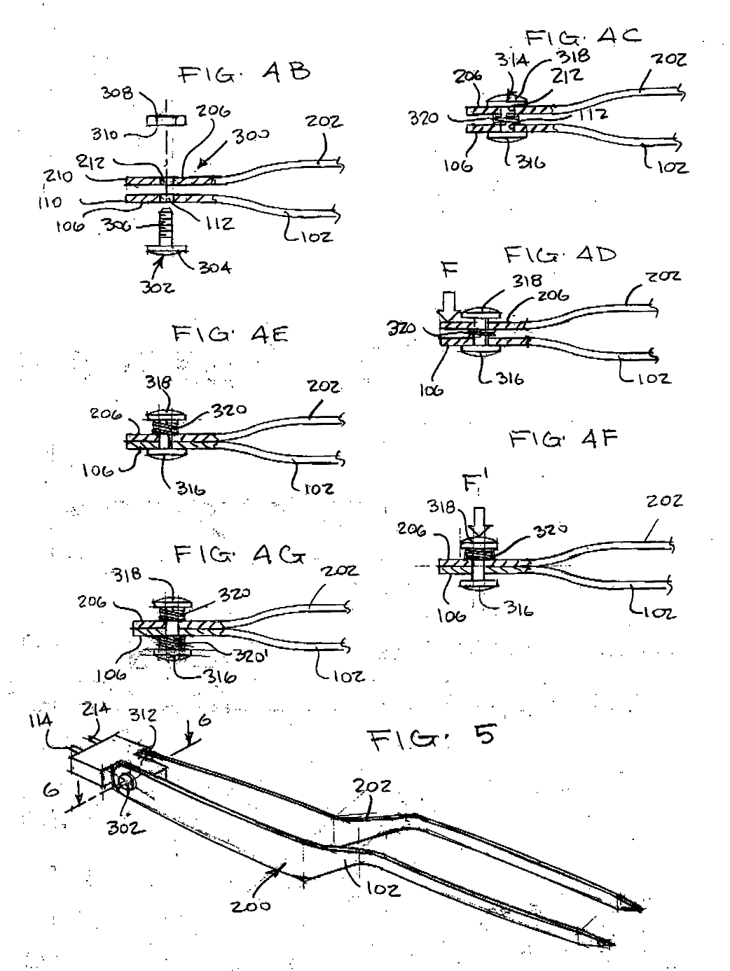

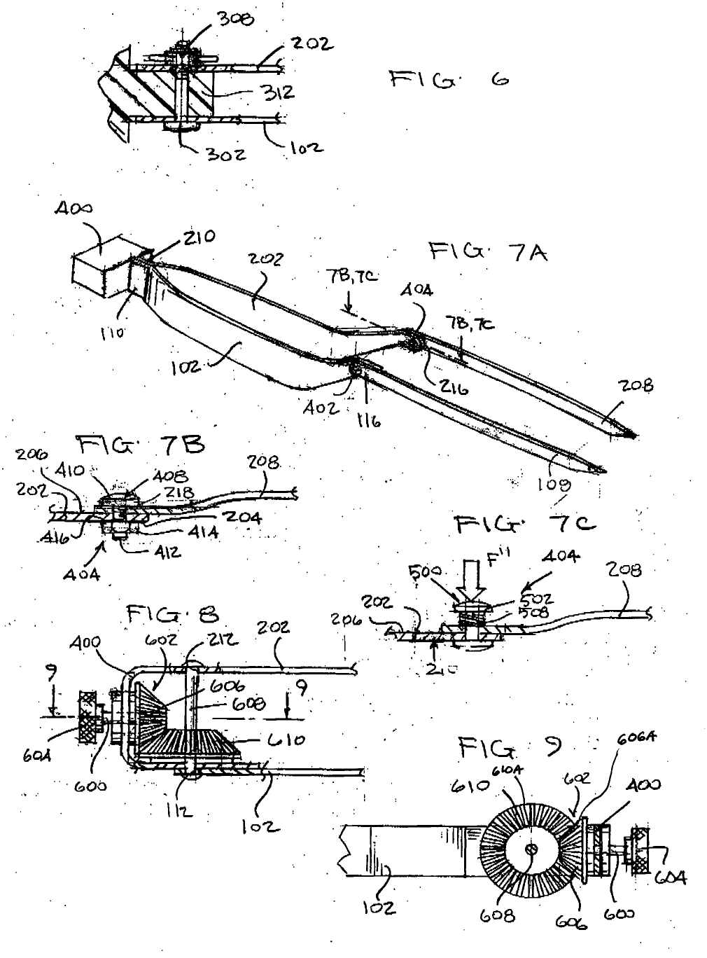

Referring now to FIGS. 3 and 4A-4E, a forceps 100 in accordance with the present invention is illustrated. The forceps 100 includes a first blade 102 and a second blade 202 both having an inner surface 104, 204, an outer surface 106, 206, a tip end 108, 208 and a proximal end 110, 210. Forceps 100 also includes a blade adjustment joint 300 disposed at the proximal ends 110, 210 of first and second blades 102, 202. As a result of joint 300 the first blade 102 and second blade 202 are rotateably connected to each other. Blade adjustment joint 300 selectively (1) allows the first and second blades 102, 202 to rotate independently of each other or (2) prevents the first and second blades 102, 202 from rotating independently of each other. Blade adjustment joint 300 permits first blade 102 to rotate or pivot in relation to second blade 202 and visa versa so the tips of forceps 100 can be realigned simply and easily. Additionally, in one condition blade adjustment joint 300 prevents first blade 102 and second blade 202 from rotating, thus locking the blades in position for use. Blade adjustment joint 300 can be manufactured to permit unrestricted rotation or to completely restrict rotation of the first blade 102 and second blade 202.

Referring now to FIGS. 4A and 4B, blade adjustment joint 300 includes a bolt 302 and a nut 308. The bolt 302 has a head end 304 and a threaded end 306. The nut 308 has a contact surface 310 which is threadably engagable with the threaded end 306 of bolt 302. Additionally, first blade 102 and second blade 202 include openings 112, 212 proximate to both of their proximal ends 110, 210. The threaded end 306 of bolt 302 passes through the first and second blade openings 112, 212 and head end 304 engages the outer surface 106 of first blade 102. Thus, when nut 308 is screwed down, threaded end 306 of bolt 302, first blade 102 and second blade 202 are prevented from rotating. Once nut 308 is loosened, both the first and second blades 102, 202 can freely rotate.

In one embodiment, contact surface 310 of nut 308 contacts and frictionally engages outer surface 206 of second blade 202 and head end 304 can frictionally engage outer surface 106 of first blade 102. The frictional engagement prevents the blades from rotating. Another embodiment prevents rotation when inner surface 104 of first blade 102 frictionally engages with inner surface 204 of second blade 202.

A further embodiment of blade adjustment joint 300, as illustrated in FIGS. 4C and 4D, includes a different bolt 314 passing through openings 112, 212. Bolt 314 has a first blade end 316 and a second blade end 318. A spring 320 is disposed on bolt 314 between the inner surfaces 104, 204 of the first and second blades 102, 202. As shown in FIG. 4C, spring 320 biases outer surface 106 of first blade 102 to frictionally engage first blade end 316 of bolt 314 and biases outer surface 206 of second blade 202 to frictionally engage second blade end 318 of bolt 314. The frictional engagement prevents the blades from rotating. The blade is released by applying a force F to either the first or second blades 102, 202, in a direction parallel to spring 320, as illustrated in FIG. 4D. The pressure disengages one of the blades from its respective blade end of bolt 314 and allows the blade to rotate.

Another spring loaded embodiment of a blade adjustment joint 300 is illustrated in FIGS. 4E and 4F, wherein spring 320 is disposed on bolt 314 between outer surface 206 of second blade 202 and second blade end 318. Spring 320 then biases inner surface 204 of second blade 202 to frictionally engage inner surface 104 of first blade 102. A force F′ is applied to either the second blade end 318 of bolt 314 or the second blade 202, in a direction parallel to spring 320. Force F′ disengages first blade 102 from first blade end 316 of bolt 314 and allows the blade to rotate. This configuration can be mirrored for first blade 102 and can be duplicated so both first blade 102 and second blade 202 are spring biased toward each other.

FIG. 4G illustrates a second spring 320′ disposed on bolt 314 between outer surface 106 of first blade 102 and the first blade end 316 of bolt 314, wherein the second spring 320′ biases the inner surface 104 of first blade 102 to frictionally engage the inner surface 204 of second blade 202. Again, when force F′ is applied to first blade 102, in a direction parallel to second spring 320′, the first blade 102 disengages from second blade 202 and can rotate with respect to it.

Forceps 100 can also be electrically charged as electro-surgical forceps 200. In this configuration, either one or both blades can be electrically charged. FIGS. 5 and 6 illustrate electro-surgical forceps 200, including at least one electrical terminal 114, 214 electrically connected to at least one of first blade 102 and/or second blade 202. Electro-surgical forceps 200 require an insulator 312 disposed between the first blade 102 and the second blade 202 to electrically the isolate the first blade 102 from the second blade 202. Insulator 312 can be any shape known in the art, including a block of insulating material or a washer. Additionally, bolt 302 and nut 308 can be manufactured from an insulating, i.e., in an electrically non-conductive material, e.g. plastic, to prevent a short circuting of the electrical circuit that charges the electro-surgical forcepts 200.

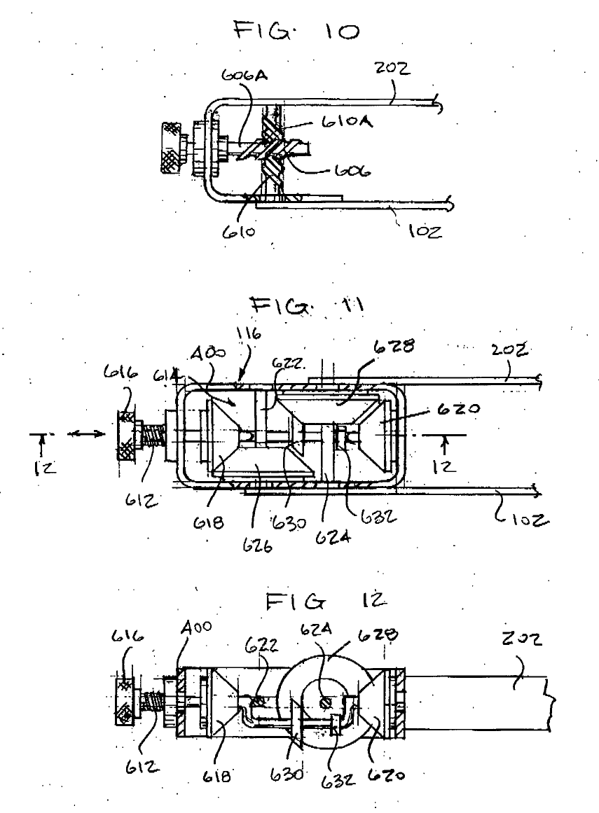

FIG. 7A, illustrates another embodiment of forceps 100. First and second blades 102, 202 include joint positions 116, 216 disposed between tip ends 108, 208 and proximal ends 110, 210. Forceps 100 further include a fixing joint 400 for preventing the rotation of proximal ends 110, 210 of the first and second blades 102, 202. Fixing joint 400 is disposed adjacement proximal ends 110, 210. A first tip adjustment joint 402 is disposed at joint position 116 of first blade 102 and a second tip adjustment joint 404 is disposed at joint position 216 of second blade 202. Tip ends 108, 208 are pivotably connected to first and second tip adjustment joints 402, 404, respectively. First and second tip adjustment joints 402, 404 selectively allow tip end 108 to pivot independent of tip end 208 and selectively prevents tip ends 108, 208 from pivoting independently of each other. In turn, adjustment joints 402, 404 selectively allow tip end 208 to pivot independent of tip end 108. First and second tip adjustment joints 402, 404 can be manufactured to permit unrestricted pivoting or completely restrict pivoting of first blade 102 and second blade 202.

First and second tip adjustment joints 402, 404 can be constructed in the same fashion as all the embodiments of blade adjustment joint 300 described above. First and second tip adjustment joints 402, 404 pivot tip ends 108, 208 in a manner similar to the way blade adjustment joint 300 pivots blades 102, 202. First and second tip adjustment joints 402, 404 can be constructed as one joint, similar to blade adjustment joint 300 or each individual tip adjustment joint 402, 404 can be constructed as a blade adjustment joint 300. For example, FIG. 7B illustrates second tip adjustment joint 404 constructed in a similar manner to blade adjustment joint 300, as illustrated in FIG. 4A. Second tip adjustment joint 404 includes a bolt 408, having a head end 410 and a threaded end 412, and a nut 414 having a contact surface 416. Second blade joint position 216 includes an opening 218. Threaded end 412 of bolt 408 passes through second blade joint position opening 218 and head end 410 engages outer surface 206 of second blade 202. Nut 414 threadably engages threaded end 412 of bolt 408. Contact surface 416 of nut 414 contacts inner surface 204 of second blade 202.

In another embodiment illustrated in FIG. 7C, second tip adjustment joint 404 includes a bolt 500 having a head end 502. A spring 508 disposed on bolt 500 wherein spring 508 biases head end 502 of bolt 500 away from outer surface 206 of second blade 202. A force F″ applied to head end 502 of bolt 500 disengages tip end 208 from proximal end 210 and allows tip end 208 to pivot. This embodiment is similar to blade adjustment joint 300 as illustrated in FIGS. 4E and 4F.

FIGS. 8 and 9 illustrate another embodiment for forceps 100, which includes a first shaft 600 rotatably disposed within fixing joint 400. Shaft 600 has an inner end 602 disposed between first and second blades 102, 202 and an opposite outer end 604. A first gear 606 is disposed on inner end 602. A second shaft 608 is fixed either to first blade opening 112 or to first joint position 116 and is rotatably disposed through second blade opening 212 or second joint position 216, and a second gear 610 is disposed on second shaft 608. Second gear 610 and first gear 606 mesh together so when outer end 604 of first shaft 600 is rotated, first gear 606 is rotated and the rotation of first gear 606 rotates second gear 610 which causes the rotation of tip end 108 of first blade 102. First gear 606 and second gear 610 can be any gear known to those of skill in the art.

FIG. 10 illustrates, for example, a worm gear 606A, 610A. Additionally, first gear 606 and second gear 610 can have coarse or fine gearing or tooth spacing 606A, 610A. Either first gear 606 and second gear 610 can be geared identically or one can be varied in relation to the other. Altering gearing 606A, 610A of first and second gears 606, 610 allows for fine or coarse adjustments of the forceps' blade. In particular, varying gearing 606A, 610A of first and second gears 606, 610 allows for a coarse movement of one gear to effect a fine movement of the other.

Another gear embodiment, illustrated in FIGS. 11 and 12, includes a first shaft 612 slidably and rotably disposed within fixing joint 400 having an inner end 614 disposed between the first and the second blades 102, 202 and an opposite outer end 616. A first gear 618 is disposed on the inner end 614 of first shaft 612 and a second gear 620 disposed on the inner end 614, opposite first gear 618. Rotation of first shaft 612 rotates first and second gears 618, 620. A second shaft 622 is fixed to either first blade opening 112 or first joint position 116. A third shaft 624 is fixed to second blade opening 212 or second joint position 216. A third gear 626 is disposed on second shaft 622 and selectively meshes with first gear 618 so that rotation of first gear 618 rotates third gear 626 which rotates tip end 108. A fourth gear 628 is disposed on third shaft 624 and selectively meshes with second gear 620 so that rotation of second gear 620 rotates fourth gear 628 which rotates tip end 216. First gear 618 and second gear 620 are arranged on first shaft 612 such that when first gear 618 and third gear 626 are meshed, second and fourth gears 620, 628 are not meshed. Conversely, when second and fourth gears 620, 628 are meshed, first and third gears 618, 626 are not meshed. The selective engagement of the gears can be accomplished in numerous ways by sizing or positioning the gears. An embodiment includes first shaft 612 sliding to alternately engage first and third gears 618, 626 or second and fourth gears 620, 628. Additionally, there can be a ‘neutral’ position where neither sets of gears are engaged or an ‘engaged’ position where both sets of gears are engaged and are secured from movement. Also, a first gear stop 630 can be disposed on first shaft 612 to prevent the rotation of the third gear 626 when first gear 618 is not meshed or to prevent the rotation of fourth gear 628 when second gear 620 is not meshed. Alternately, a second gear stop 632 can be disposed on first shaft 612 to prevent the rotation of fourth gear 628 when second gear 620 is not meshed.

As above, first, second, third, and fourth gears 618, 620, 626, 628 include a plurality of teeth and numerous arrangements of teeth and gears are contemplated. A basic embodiment is where the spacing of the plurality of teeth on all the gears is the same. Other embodiments include first and third gears 618, 626 including a first spacing of a plurality of teeth, and second and fourth gears 620, 628 include a second spacing of a plurality of teeth, where the first spacing of the teeth does not equal the second spacing of the teeth. This allows for one blade to have a coarse adjustment and one blade to have a fine adjustment.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, and maybe merely conceptual in nature. It is the intention, therefore, to be limited only as indicated by the scope of the claims appended hereto.

1. A forceps comprising:
a first blade having an inner surface, an outer surface, a tip end and a proximal end;
a second blade having an inner surface, an outer surface, a tip end and a proximal end;
a blade adjustment joint disposed proximate to the proximal end of the first and the second blades;
the first blade being rotateably connected to the blade adjustment joint, wherein the blade adjustment joint selectively allows the first blade to rotate independently of the second blade and selectively prevents the first blade from rotating independently of the second blade.

2. The forceps of claim 1, wherein the blade adjustment joint comprises:
a bolt having a head end and a threaded end;
a nut having a contact surface;
the first blade includes an opening proximate to the proximal end;
the second blade includes an opening proximate to the proximal end;
the bolt being adapted to pass the threaded end through the first blade opening and the second blade opening and the head end engages the outer surface of the first blade;
the nut threadably engagable with the threaded end of the bolt; and
the contact surface of the nut contacting the outer surface of the second blade.

3. The forceps of claim 2, wherein the contact surface of the nut frictionally engages the outer surface of the second blade and the head end of the bolt frictionally engages the outer surface of the first blade.

4. The forceps of claim 2, wherein the inner surface of the first blade frictionally engages with the inner surface of the second blade.

5. The forceps of claim 2, further comprising:
at least one electrical terminal electrically connected to at least one of the first blade and the second blade; and
an insulator disposed between the first blade and the second blade to electrically isolate the first blade from the second blade.

6. The forceps of claim 1, wherein the blade adjustment joint comprises:
a bolt having a first blade end and a second blade end;
the first blade includes an opening proximate to the proximal end;
the second blade includes an opening proximate to the proximal end;
the bolt being adapted to pass through the first blade opening and the second blade opening;
a spring disposed on the bolt between the inner surfaces of the first and the second blades, wherein the spring biases the outer surface of the first blade to frictionally engage the first blade end and biases the outer surface of the second blade to frictionally engage the second blade end; and
wherein a pressure applied to one of the first and the second blades, in a direction parallel to the spring, comprises the spring, disengages one of the first blade and the second blade from the respective first and second blade end.

7. The forceps of claim 6, further comprising:
at least one electrical terminal electrically connected to at lease one of the first blade and the second blade; and
an insulator disposed between the first blade and the second blade to electrically isolate the first blade from the second blade.

8. The forceps of claim 1, wherein the blade adjustment joint comprises:
a bolt having a first blade end and a second blade end;
the first blade includes an opening adjacent to the proximal end;
the second blade includes an opening adjacent to the proximal end;
the bolt being adapted to pass through the first blade opening and the second blade opening;
a spring disposed on the bolt between the outer surface of the first blade and the first blade end wherein the spring biases the inner surface of the first blade to frictionally engage the inner surface of the second blade; and
wherein a pressure applied to the first blade, in a direction parallel to the spring, comprises the spring and disengages the first blade from the second blade.

9. The forceps of claim 8, further comprising:
at least one electrical terminal electrically connected to at least one of the first blade and the second blade; and
an insulator disposed between the first blade and the second blade to electrically isolate the first blade from the second blade.

10. The forceps of claim 8, wherein the blade adjustment joint further comprises:
a second spring disposed on the bolt between the outer surface of the second blade and the second blade end, wherein the second spring biases the inner surface of the second blade to frictionally engage the inner surface of the first blade; and
wherein a pressure applied to the second blade, in a direction parallel to the second spring, compresses the spring and disengages the second blade from the first blade.

11. The forceps of claim 10, further comprising:
at least one electrical terminal electrically connected to at least one of the first blade and the second blade; and
an insulator disposed between the first blade and the second blade to electrically isolate the first blade from the second blade.

12. A forceps comprising:
a first blade having an inner surface, an outer surface, a tip end, a proximal end and a first joint position disposed between the tip end and the proximal end;
a second blade having an inner surface, an outer surface, a tip end a proxiaml end and a second joint position disposed between the tip end and the proximal end;
a fixing joint for preventing the rotation of the distal ends of the first and second blades disposed adjacent the proximal end of the first and the second blades;
a first tip adjustment joint disposed at the first and second joint positions; and
the tip ends of the first blade pivotably connected to the first tip adjustment joint, wherein the first tip adjustment joint selectively allows the tip end of the first blade to pivot independent of the second blade and selectively prevents the tip end of the first blade from pivoting independent of the second blade.

13. The forceps of claim 12, wherein the tip adjustment joint comprises:
a bolt having a head end and a threaded end;
a nut having a contact surface;
the first blade joint position includes an opening adjacent to the proximal end;
the second blade joint position includes an opening adjacent to the proximal end;
the bolt being adapted to have the threaded end pass through the first blade opening and the second blade opening, and the head end being adapted to engage the outer surface of the first blade;
the nut being threadably engagable with the threaded end of the bolt; and
the contact surface of the nut contacting the outer surface of the second blade.

14. The forceps of claim 13, wherein the contact surface of the nut frictionally engages the outer surface of the second blade and the head end of the nut frictionally engages the outer surface of the first blade.

15. The forceps of claim 13, wherein the inner surface of the first blade frictionally engages with the inner surface of the second blade.

16. The forceps of claim 13, further comprising:
at least one electrical terminal electrically connected to at least one of the first blade and the second blade; and
an insulator disposed between the first blade and the second blade to electrically isolate the first blade from the second blade.

17. The forceps of claim 12, further comprising:
a second tip adjustment joint;
wherein the first tip adjustment joint is disposed at the first joint position and the second tip adjustment joint is disposed at the second joint position.

18. The forceps of claim 12, wherein the tip adjustment joint comprises:
a first shaft rotably disposed within the fixing joint and having an inner end disposed between the first and the second blades and an opposite outer end;
a first gear disposed on the inner end of the first shaft;
a second shaft fixed to the first joint position and rotatably disposed through the second joint position; and
a second gear disposed on the second shaft and meshing with the first gear, wherein rotating the outer end of the first shaft results in rotating the first gear, the rotation of the first gear rotates the second gear and rotates the tip end of the first blade.

19. The forceps of claim 18, wherein the first gear and the second gear are worm gears.

20. The forceps of claim 12, wherein the tip adjustment joint comprises:
a first shaft slidably and rotably disposed within the fixing joint and having an inner end disposed between the first and the second blades and an opposite outer end;
a first gear disposed on the inner end of the first shaft;
a second gear disposed on the inner end of the first shaft and opposite the first gear, wherein rotating the first shaft rotates the first and the second gears;
a second shaft fixed to the first joint position
a third shaft fixed to the second joint position;
a third gear disposed on the second shaft and selectively meshing with the first gear, wherein rotating the first gear rotates the third gear and rotates the tip end of the first blade;
a fourth gear disposed on the third shaft and selectively meshing with the second gear, wherein rotating the second gear rotates the fourth gear and rotates the tip end of the second blade and, wherein when the first and the third gears are meshed, the second and the fourth gears are disengaged, and when the second and the fourth gears are meshed, the first and the third gears are disengaged.

21. The forceps of claim 23, wherein the tip adjustment joint further comprises:
a first gear stop disposed on the first shaft to prevent the rotation of the third gear when the first gear is disengaged; and to prevent the rotation of the fourth gear when the second gear is disengaged.

22. The forceps of claim 23, wherein the tip adjustment joint further comprises:
a first gear stop disposed on the first shaft for preventing the rotation of the third gear when the first gear is disengaged; and
a second gear stop disposed on the first shaft for preventing the rotation of the fourth gear when the second gear is disengaged.

23. The forceps of claim 20, wherein the first, the second, the third, and the fourth gears have a plurality of teeth with a particular spacing, and wherein the spacing of the teeth on the first gear equals the spacing of the teeth on the second, the third and the fourth gears.

24. The forceps of claim 20, wherein the first and the third gears have a plurality of teeth with a first spacing, and the second and the fourth gears having a plurality of teeth with a second spacing, wherein the first spacing of the teeth does not equal the second spacing of the teeth.
